(19) United States
(12) Patent Application Publication
(10) Pub. No.: US 2008/0125810 A1
(43) Pub. Date: May 29, 2008

(54) Encased tweezers

(76) Inventor: Yong Hoon Cho, Fullerton, CA (US)

Correspondence Address: Maria Erlinda C. Sarno, Esq., P.O. Box 1023, Artesia, CA, 90702, US

(21) Appl. No.: 11/605,631

(22) Filed: Nov. 28, 2006

(51) Int. Cl. A61B 17/50

(52) U.S. Cl. 606/211

(57) ABSTRACT

An encased tweezers with a trough like hollow case having a hollow trough section constricting on one end, the hollow trough section partially concealing a body of the tweezers and allowing the head of the tweezers to be viewed from the top when the tweezers is in a nested position. The tweezers has a fused end and a pincer opposite the fused end. The fused end of the tweezers insert between two opposite borders at the constricted end of the hollow case where the tweezers is attached to the hollow case. A positional plate between an inside surface of one border and a lateral side of the tweezers or between adjacent lateral sides of two tweezers can be used for positioning the tweezers when it is deployed. A mechanism for ejecting the tweezers from the hollow case is also described.

This invention relates to a tweezers or multiple tweezers having an encased or covered tip for safety and versatility of usage.

### BACKGROUND

Current tweezers are usually marketed as is, that is, without any cover for the tips which can potentially cause personal injury or damage to a container susceptible to scratching or tearing. One solution offered is to encase the whole tweezers in a container. While this may solve the safety issue, one will find it cumbersome and time consuming to open the container every time a tweezers is needed. Also, with usage, one will likely lose the container, especially the cover, when the tweezers is not returned to the container after each usage. Also, for multiple tweezers with multiple heads, multiple containers are needed which would require some type of an identification to differentiate one tweezers from the other. Otherwise, one has to open each container until the right tweezers is found.

Tweezers now come with different heads because these have found multiple usage and application. There are those used for cosmetic purposes, usually for plucking unwanted hair, there are those used in surgery for holding or gripping tissues, blood vessels, organs and the like, there are those used in dentistry for clamping a tooth, there are those used in weaving and embroidery for plucking or inserting a thread, etc. Because of these varied usage, the tweezers come in different forms of heads. Head in this application is used to denote the pincer portion of the tweezers with its tips.

The body of an ordinary tweezers is generally constructed from two identical elongated thin flat sheets. The two elongated flat sheets are fused together at one end to form the stationary end resulting in a pair of free opposing ends. The pair of free opposing ends opposite the fused end having the tips are referred to herein as the pincer. The fusion at one end causes the opposite end to open up or space apart, consequently, allowing movement at this end as pressure by the fingers for example, is exerted or released at the outside surfaces of the elongated flat sheets proximal to the free end, herein referred to as pincer.

It is therefore an object of this invention to provide a case for a tweezers that will cover the tip without masking the identity of its head.

It is also an object of this invention to provide a case that will hold multiple tweezers for variable purposes or functions and cover but not mask its heads or tips for easy identification and retrieval of a desired tweezers.

It is also an object of this invention to provide a case that can deploy a given tweezers at variable positions.

It is also a further object of this invention to provide a case that is attached to the tweezers.

It is still a further object of this invention to provide tweezers with ergonometrically designed body instead of the conventional thin flat elongated sheets.

It is also still a further object of this invention to provide a simple mechanism for ejecting a tweezers from the case.

### SUMMARY

This invention relates to a trough like hollow case for a tweezers or multiple tweezers that covers the tweezer's tip while the tweezers is not in use. The encased tweezers comprises a trough like hollow case having a hollow trough section constricting on one end and a tweezers having a fused end and a pincer opposite the fused end, the fused end inserting between two opposite borders at the constricted end of the hollow case. The hollow trough section partially conceals the body of the tweezers, leaving a top portion protruding beyond a top border of the hollow case when the tweezers is in a nested position. The protruding portion provides a grip space for deploying the tweezers from the hollow case. The tweezers' head can be viewed from the outside, above the case, because the hollow case is shallow and wide. The tweezers attached to the hollow case can have a variety of heads or tips. One means for attaching the tweezers to the hollow case is by a pin or a screw going through aligned openings on the fused end of the tweezers and the hollow case. The body of the tweezers can have curved or flat lateral sides and peripheral edges. The pincers should be made of a resilient material to allow easy closure and opening of the tips. One or both outside surfaces of the borders at the constricted end of the hollow case can be capped. To assist in deploying the tweezers at different positions or angles, a polymeric ring is placed inside an etched out circular cavity around an opening on a lateral side of the fused end of the tweezers where the tweezers connect with the hollow case. The polymeric ring/s provide frictional hold on the tweezers thereby preventing them from unintentionally nesting at the hollow case. Another way of achieving this is through the introduction of a positional plate between an inside surface of one border and a lateral side of the tweezers or between adjacent lateral sides of two tweezers. The positional plate has a front face and a back face, the front face having a triangularly curved elevation/s positioned at a desired angle along an outside surface of the positional plate. There is a mole on top of the elevation. This mole lodges on an indentation on the tweezers, keeping the tweezers at the angle where the mole is located. The tweezers can be deployed manually or by ejecting the tweezers from the hollow case. This can be achieved by a combination of a curved spring placed inside a bottom surface of the hollow case and a latch having a switching head on one end and a staging seat on the other end introduced into an opening with a recessed protruding portion at a bottom end of the hollow trough section. The recessed protruding portion engages with the switching head of the latch as it traverse away from the constricted end of the hollow trough when a tip of the tweezers is rested on the staging seat as the tweezers press on the curved spring keeping the tweezers nested inside the hollow trough. When the switching head is moved towards the constricted end, the staging seat retracts and releases the tip of the tweezers allowing the curved spring to relax, thereby eject the tweezers from the hollow case.

Other embodiments of the present invention will become readily apparent to those skilled in the art from the following detailed description, wherein it shows and describes only certain embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 is a top plan view of a case holding one tweezers at its nested or closed position.

FIG. 1A is a perspective view of a case holding one tweezers at its deployed or open position.

FIG. 2 is a top plan view of a case holding two tweezers at its nested or closed position.

FIG. 2A is a perspective view of a case holding two tweezers, one at its deployed or open position and the other at its nested or closed position.

FIG. 2B is a perspective view of an encased one tweezers showing in detail the outside surface of the back border of the case.

FIG. 3 is an exploded view of the components of the encased tweezers with one tweezers.

FIG. 3A is an exploded view of the components of the encased tweezers with two tweezers.

FIG. 3B is a cross sectional view of the case taken along 1-1 of FIG. 2B showing the inside surface of the front border of the case.

FIG. 3C is an exploded view of the components of the encased tweezers with four tweezers.

FIG. 4A is a side view of the front border of the encased tweezers.

FIG. 4B is a side view of the back border of the encased tweezers.

FIG. 5 is a back perspective view of the bottom section of the case showing an opening for a latch.

FIG. 6 is an exploded view of the components of an encased tweezers having additional parts for easy deployment of the tweezers and for multi-positioning the tweezers at given angles.

FIG. 7 is a bottom view of the case showing two posts for holding a spring.

FIG. 7A is a bottom view of the case having a spring installed at its inner surface with the tip of the posts flattened.

FIG. 8 is an enlarged side view of a latch.

FIG. 9 is a sectional view showing the latch of FIG. 8 restraining a tweezers and a spring by lodging the tweezers tip at a lateral staging seat of the latch.

FIG. 9A is a sectional view showing the latch of FIG. 8 ejecting the tweezers and releasing the spring by dislodging the tweezers tip from the lateral staging seat of the latch.

FIG. 10 is a perspective view of an encased tweezer having a positional plate in place.

FIG. 11A is a front plan view of a positional plate.

FIG. 11B is a back plan view of a positional plate.

FIG. 12 is a perspective view of an encased multiple tweezers having positional plates.

FIG. 13 is an exploded view of the components of an encased two tweezers with positional plates.

FIG. 14 is an exploded view of the components of an encased four tweezers with one way of arranging the positional plates.

FIG. 14A is an exploded view of the components of an encased four tweezers with another way of arranging the positional plates.

### DETAILED DESCRIPTION OF THE INVENTION

The detailed description represented herein is not intended to represent the only way or the only embodiment in which the claimed invention may be practiced. The description herein is provided merely as an example or examples or illustrations of the claimed invention and should not be construed as the only way or as preferred or advantageous over other embodiments or means of practicing the invention. Any tweezers housed in a case protecting but not masking its head or tip is within the scope of this invention. The detailed description includes specific details to provide a thorough understanding of the claimed invention and it is apparent to those skilled in the art that the claimed invention may be practiced without these specific details. In some instances, well known structures and devices are shown in block diagrams or drawn with broken lines in order to either avoid obscuring the main concepts of the invention or to show the relationship of one part to the other.

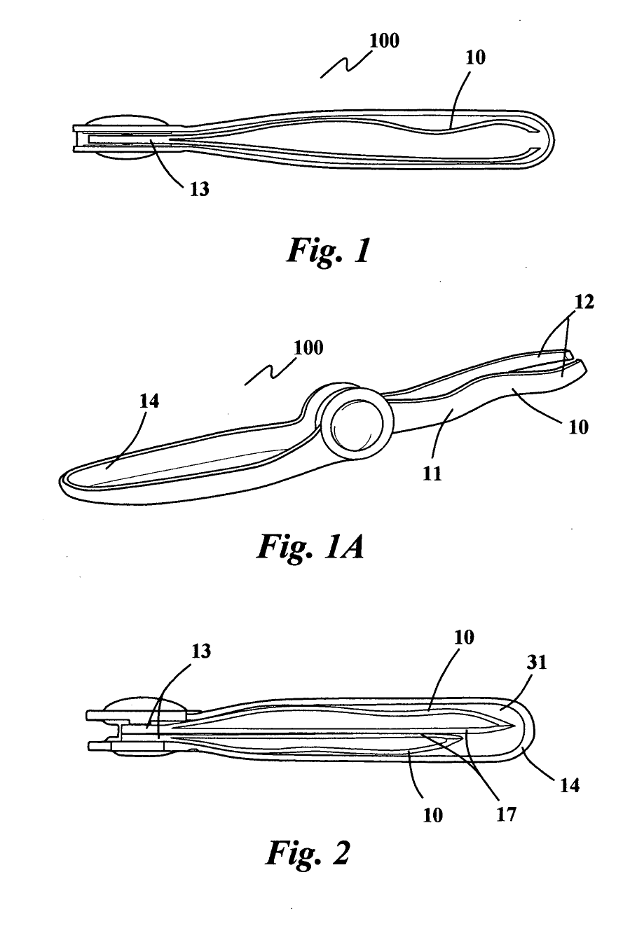

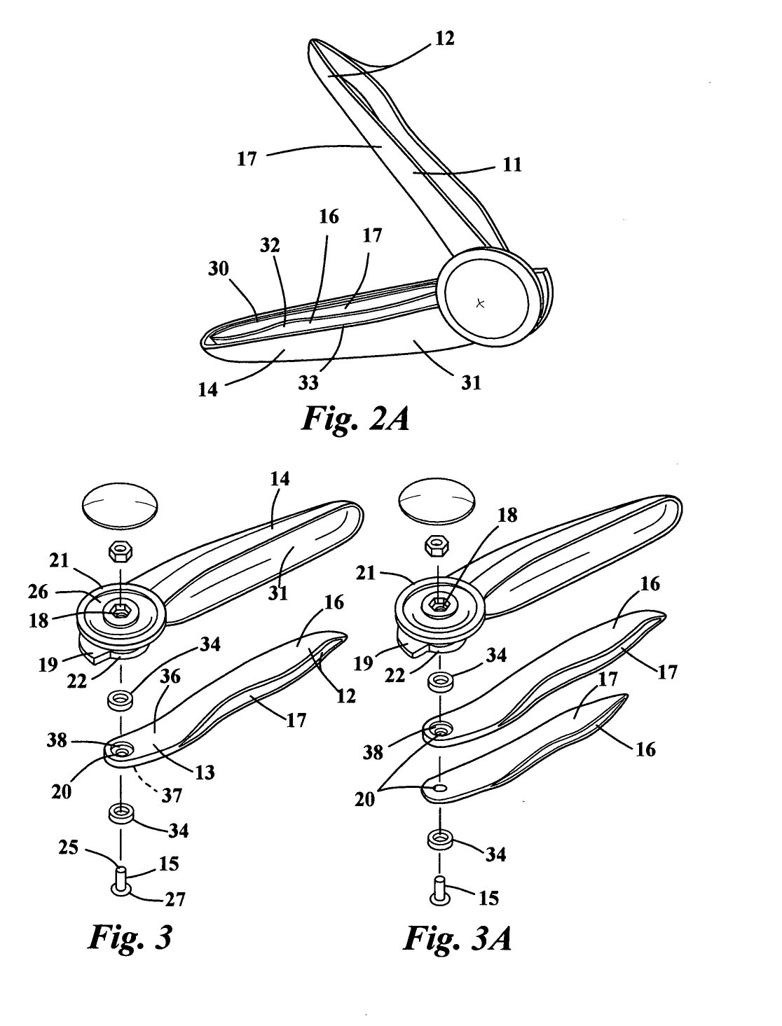

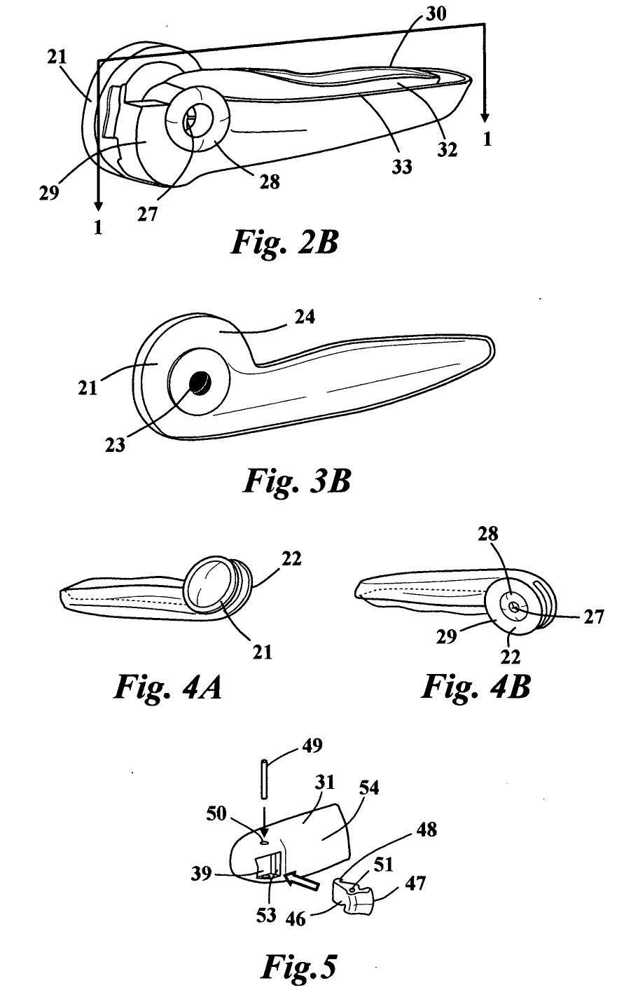

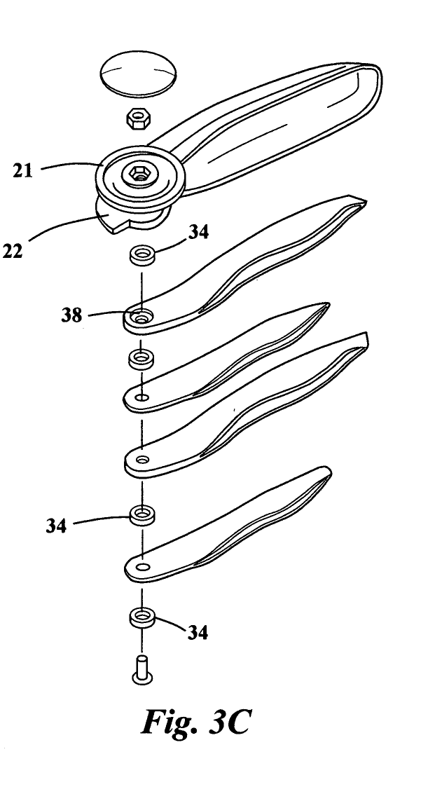

FIGS. 1 and 1A are views of an encased tweezers 100 having one tweezers 10, FIG. 1 in its nested or closed position and FIG. 1A in its deployed or open position. The body 11 of the tweezers include a pair of elongated arms or pincers 12 on an end opposite a fused end 13. FIGS. 1 and 1A show one tweezer while FIGS. 2 and 2A show two tweezers with the case 14. FIG. 3 shows the components of the covered or encased tweezers in its simplest form. The case 14 is trough like constricting on one end where the fused end of the tweezers are held by a pivot pin or screw 15 as shown in FIGS. 2B, 3, 3A, 3C and 4B. If there is only one tweezers inside the case 14, the arms or pincers of the body 11 can both have curved lateral sides 16. For multiple tweezers as shown in FIGS. 2 and 2A, each tweezers will have at least one straight or flat lateral side 17 facing or adjacent to each other.

In its simplest form, one tweezers 10 is held inside case 14. Case 14 has a straight through hole 18 on the constricted end 19 for accommodating a screw or a pivot pin 15. To encase a tweezers, a hole 20 is bored on the fused end 13 of the tweezers and the fused end is then introduced into the constricted end 19 between the two borders of the case 14, a front border 21 and a back border 22, with the hole 20 aligning with the hole 18 of the case 14 bored at both borders 21 and 22. The front border 21 instead of having a hole can just have an etching or indentation 23 at its inside surface 24 as shown in FIG. 3B matching the contour of the top end 25 of the screw or pin 15 where the top end 25 will enter and rest after interconnecting the case with the tweezers. The outside surface 26 of the front border 21 at the constricted end 19 may be capped as shown more clearly in FIG. 4A to give an aesthetically desirable look and conceal one end of the screw or pin 15. One convenient way of capping this is by snapping the cap over the surface to be covered. Other means of capping can also be implemented. On the other border 22, the head 27 of the screw 15 is somewhat concealed by making the head 27 advance inwards of a dome like elevation 28 on the outside surface 29 of the back border 22 as shown in FIGS. 2B and 4B. The identification of which is the front border and the back border is arbitrary. Its designation is merely to facilitate the relationship between the text and the drawings provided. Depending on the view shown, these may also be labeled upper or lower border.

To use the tweezers, one simply holds a top lateral edge 30 of the tweezers and pulls the tweezers out. This is possible because the hollow trough section 31 of the case 14 opposite the constricted end 19 only partially conceals the lateral sides 16 and 17 of the pincers, allowing a top portion 32 to protrude beyond the top border 33 of case 14. As can be seen on FIGS. 1 and 2, the case provides ample space for the tweezers, that is, the tweezers are not squeezed inside the case. This also allows easy deployment of the tweezers. The case is also shallow with the walls of the hollow trough extending at a height just enough to cover the tip/s of the tweezers. Consequently, a user can see the tip of the tweezers when viewed from the top of the hollow case. This enables the user to deploy the desired tweezers in a multiple encased tweezers without trial and error. The extent or the area of the protruding portion of the tweezers provides enough gripping space to pull the tweezers easily out of the case.

For an encased two or more tweezers' heads, it is recommended to have a rubber or rubber like polymeric ring 34 such as a silicone ring between the inside surfaces 24 and 35 of the borders and the outside surfaces 36 and 37 of the fused lateral sides of the tweezers. To ensure that the ring 34 stay at its desired location, a circular cavity 38 matching the contour of the ring is etched out around the hole 20 on the tweezers. On an encased two tweezers' heads, this cavity 38 is usually on one of the fused lateral sides of the tweezers, the side facing the inner surfaces, 24 and 35, of the front and back borders. There is usually no cavity or ring needed on the lateral sides of the tweezers facing each other as shown in FIG. 3A. However, the cavity-ring combination can also be placed between them, if one desires to do so. FIG. 3 shows how this can be done. FIG. 3C employs both ways of installing the ring. These rings facilitate the removal or deployment of the tweezers and vice versa and more importantly, allow one to deploy the desired tweezer at any desired angle because the ring will provide the necessary friction to keep the tweezers from moving out of position as shown in FIG. 2A or returning to its nested position unintentionally. The degree of tightness in fastening the tweezers with the case can also be adjusted because of the presence of the ring/s. FIGS. 2 and 2A show how the case holds two tweezers. For the encased two tweezers with rings 34, the rings are placed in their respective cavities 38 and the two tweezers are placed side by side and introduced, in the same manner as the one encased tweezers, between the two borders 21 and 22. In the illustration shown in FIG. 3A, the tweezers are held to the case by a screw and a nut, the screw going through the aligned holes 18 and 20. The outside surface of the front border is likewise capped for aesthetic look and for covering the tips of the screw or pin for safety. Like the encased one tweezer head, the tweezers are deployed or pulled out from the case manually by holding on the exposed protruding top portion 32 of the tweezers. Having two tweezers instead of one, we can have two heads available at all times. While there are only two tweezers shown here, it is so easy to construct more than two tweezers by simply widening the hollow trough section 31 and the distance between borders 21 and 22 of the case 14 and placing a ring 34 on additional circular cavities bored around the respective holes on the lateral sides of the tweezers facing each other as shown in FIG. 3C. Examples of heads or tips of the tweezers are square, pointed, slanted, curved, serrated, toothed, ringed, etc. depending on the intended usage of the tweezers. As shown in the drawings, it is also recommended to ergonomically curve the finger contacting lateral side 16 of the tweezers instead of being straight and flat.

Materials that are currently used to make tweezers can be used for this claimed invention such as metal or metal alloys like stainless steel, titanium, chromium and others. Plastics have also been used for manufacturing tweezers. A required characteristic is to have resiliency on the pincers in order to manually operate the pincers between an open (releasing the object) and closed position (clamping the object). A user grasps the body of the tweezers anywhere along the longitudinal axis of the pincers and the pressure exerted on the tips will depend upon the distance of the tips from the source of the pressure. The curved surface also offers a more aesthetic impression. A curved or concaved pincer provides a feeling of greater grip to the user. The lateral sides 16 and 17 on the pincer section of the tweezers is also recommended to have a wider surface to provide more room and allow the fingers to exert more pressure on the surfaces without hurting the fingers. The bottom peripheral edges opposite the respective peripheral top edges of the pincers 12 can be shaped the same or differently than the peripheral top edges as shown in FIG. 2A.

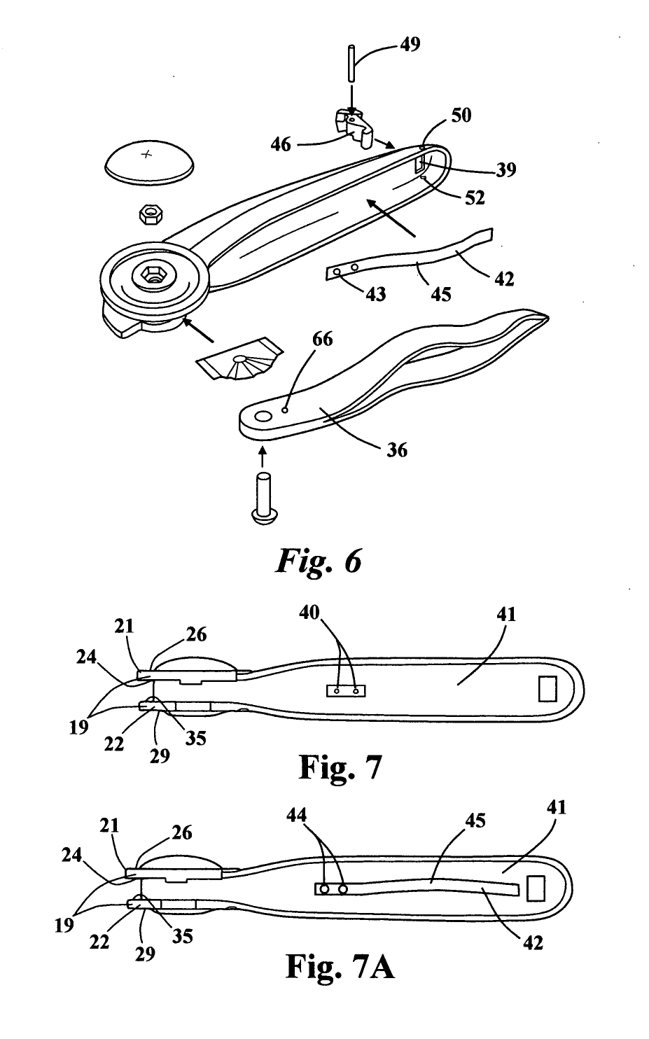

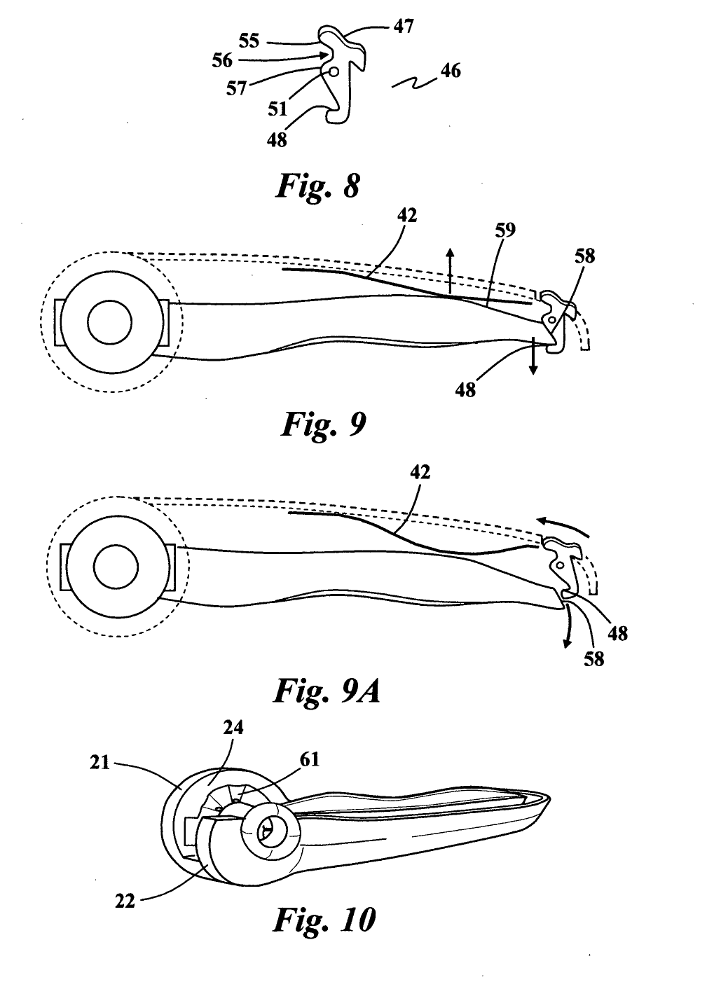

Deploying and nesting the tweezers manually requires a user to pull or push the desired tweezers in or out of the case against the tendency of the tweezers to remain in its position. To address this, certain modification and additional parts are introduced in to the case 14. An opening 39 is bored on a bottom end of the hollow trough section 31 opposite the constricted end 19. Further, a post or posts 40 are molded integrally within the inside bottom surface 41 of the case as shown in FIG. 7. A curved spring 42 having holes 43 for accommodating the post/s 41 are placed on the inside bottom surface 41 of the case by inserting the post/s into the hole/s 43. The placement of the spring is made permanent by heating the top exposed end 44 of the posts protruding from the top surface of the spring to melt or soften the end so it can be flattened into a head having a diameter larger than the diameter of the holes 43 of the spring, thereby disabling the spring from being pulled out of the post/s 40 as shown in FIG. 7A. The spring is concave shaped as shown in FIG. 6. The spring after installation on the inside bottom surface of the case does not extend into the opening 39 as shown in FIG. 7A. The concave surface 45 of the spring 42 situates near the pincer portion of the tweezers when in a nested position. A latch 46 is inserted into opening 39 on the bottom end of the trough section 31 as shown in FIGS. 5 and 6 with the switching head 47 covering the opening 39. The end opposite the switching head having a lateral staging seat 48 enters the interior of the hollow trough section 31. FIG. 8 shows an example of a design for the latch 46. The latch connects to the case through a pin 49 introduced into another hole 50 on one border of the case 14 proximal to the bottom end opposite the constricted end 19. The pin 49 enters into this hole 50 then through the hole 51 on the latch and out, lodging into a matching circular indentation 52 on the inside surface of the second border which holds and secures the pin in place. The opening 39 has a lateral protruding portion 53 recessed from the bottom outside surface 54 of the case as shown in FIG. 5. The lateral protruding portion 53 engages with the front bottom surface 55 of the switching head 47 of the latch 46 and situates on the space 56 between the switching head and a curved section 57 of the latch. The lateral protruding portion 53 also provides a platform when the switching head 47 traverse towards or away from the constricted end 19 of the case.

The tip 58 of the tweezers, when in a nested position, is rested on top of the lateral staging seat 48 at the bottom end of latch 46. When the tweezers is at this position, the lateral edges 59 of the tweezers press and strain the spring 42 as shown in FIG. 9. To deploy the tweezers, the switching head is pushed towards the constricted end 19 of the case. This movement causes the lateral staging seat 48 to retract and release the tip 58 from the seat 48. Once the tip 58 is released, the spring is likewise released and relaxed from the strained position thereby pushing or ejecting the tweezers from the case as shown in FIG. 9A.

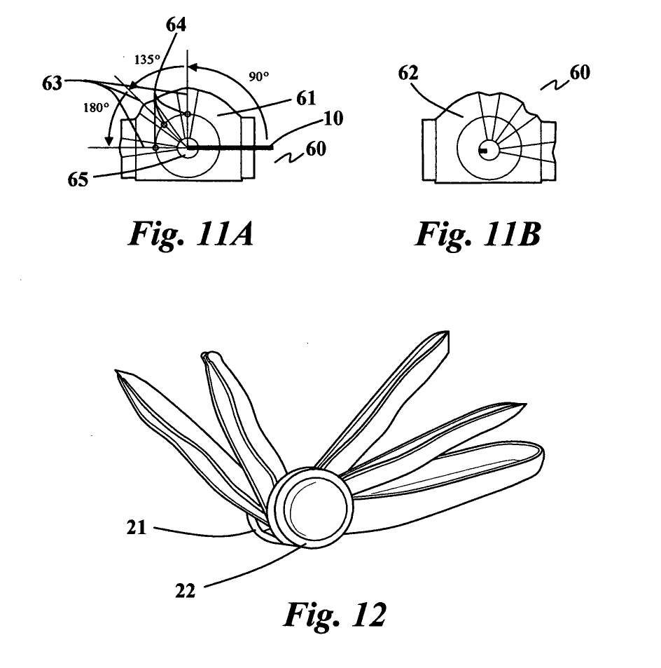

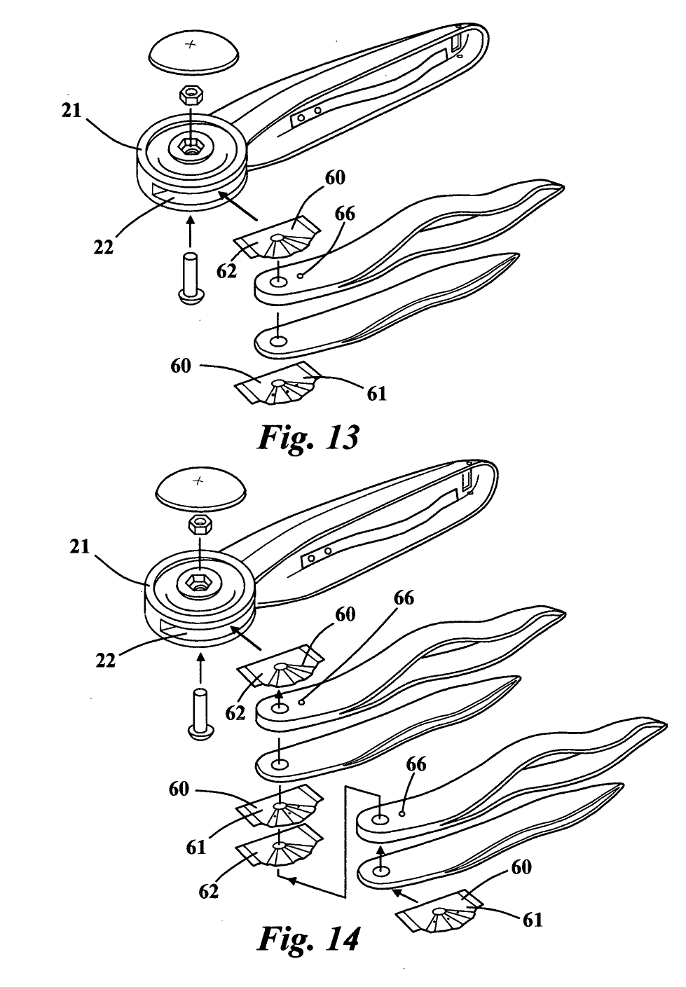

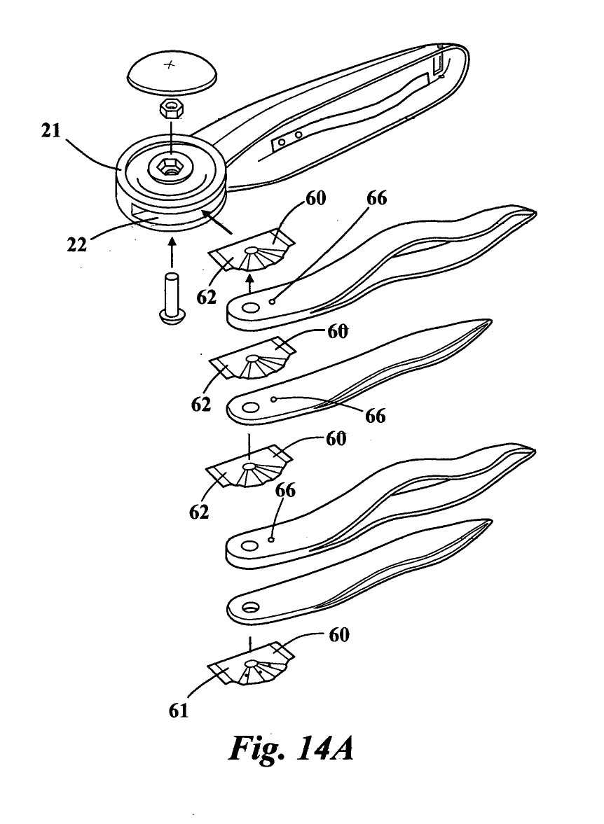

Instead of a ring causing frictional hold on the tweezers while it is deployed at different angles from 0 to 180 degrees, a positional plate 60 may be placed between the inside surface 24 of the border 21 and the lateral side 36 of the fused end of the tweezers (also referred to as fused lateral side) tweezers as shown in FIGS. 6 and 10. The positional plate 60 has a front face 61 and a back face 62 as shown in FIGS. 11A and 11B. As shown in FIG. 10, the front face 61 faces the fused lateral side 36 of the tweezers after the tweezers is introduced into the case 14. The front face has in this illustration, three triangularly curved elevation 63 with a mole 64 on top of the elevations located more proximal to an opening 65 of the positional plate 60. The elevation shows the mole 64 at the 90, 135 and 180 degrees position with the 180 degrees as the angle where the tweezers will be fully deployed. The location of the elevations and the moles need not be at these designated angles but they can be designed to fit other angles of choice. When a positional plate is used, the fused lateral side 36 of the tweezers has a matching indentation 66 where the mole 64 would lodge. The tweezers temporarily locks at the desired angle by lodging the mole positioned at the desired angle on the positional plate into the matching indentation on the tweezers. For example, if a user wants to deploy a tweezers at a 90 degree angle, it is the mole at the 90 degree position that would be lodged into the indentation 66 at the fused lateral side of the tweezers. The triangular elevations 63 have sloping sides to facilitate the dislodgment of the mole 64 from the indentation 66 by a slight pull followed by a rotational movement of the tweezers to the next position. The back face of the positional plate is shown in FIG. 11B. Although FIG. 6 shows only one tweezers, the case can be easily modified by merely adding more positional plates and tweezers and widening the bottom surface of the case 14. For example, to accommodate two tweezers, one positional plate is placed adjacent to one border, with the front face facing a fused lateral side of a tweezer having a matching indentation for the moles on the front face of the positional plate while another positional plate is placed adjacent to the other border with the front face also facing a fused lateral side of a second tweezers having a matching indentation for the moles on the front face of the second positional plate. FIG. 13 shows how the parts stack together. As in the one tweezers, the two tweezers are placed between these two positional plates which are then all interconnected with the case through a screw or pin inserted into the aligned holes of the case, the positional plates, and the tweezers, at the constricted end of the case. The outside surfaces of the front and back borders may be identically designed, that is, capped at both outside surfaces (compare back border 22 in FIG. 10 from the back border 22 in FIG. 12 to visualize). In a multiple encased tweezer, it is recommended to cap the outside surfaces of the front and back borders identically after the pins or screw have joined the tweezers with the positional plates inside the case 14 as shown in FIG. 12. To add more tweezers, additional plates between each tweezers or two positional plates in a back to back orientation, that is, exposing the moles on both front faces on the exterior, is placed between the tweezers and both lateral sides of these tweezers are provided with the matching indentation for lodging or engaging with the moles on the front face of the positional plate. FIGS. 14 and 14A show how a four tweezers will stack together inside a case.

While the embodiments of the present invention have been described, it should be understood that various changes, adaptations, and modifications may be made therein without departing from the spirit of the invention and the scope of the claims.

I claim:

1. An encased tweezers, comprising:
a trough like hollow case having a hollow trough section constricting on one end, the hollow trough section partially concealing a body of the tweezers, leaving a top portion protruding beyond a top border of the hollow case when the tweezers is in a nested position, the protruding portion providing a grip space for deploying the tweezers from the hollow case;
a tweezers having a fused end and a pincer opposite the fused end, the fused end inserting between two opposite borders at the constricted end of the hollow case; and,
means for attaching the tweezers to the hollow case.

2. The encased case of claim 1 wherein the tweezers is more than one.

3. The encased case of claim 1 wherein the tweezers attached to the hollow case have a variety of tips.

4. The encased case of claim 1 wherein the means for attaching the tweezers to the hollow case is a pin or a screw going through aligned openings on the fused end of the tweezers and the hollow case.

5. The encased case of claim 1 wherein the body of the tweezers have curved or flat lateral sides and peripheral edges.

6. The encased case of claim 1 wherein the hollow case is shallow and wide enabling a user to see a head of the tweezers when viewed from the top of the hollow case.

7. The encased case of claim 1 wherein one or two borders at the constricted end of the hollow case have a capped outside surface.

8. The encased case of claim 1 wherein the tweezers has an etched out circular cavity around an opening on a lateral side of the fused end for accommodating a polymeric ring.

9. The encased case of claim 1 wherein the pincers are made of a resilient material.

10. The encased case of claim 1 further comprising a positional plate between an inside surface of one border and a lateral side of the tweezers or between adjacent lateral sides of two tweezers for positioning the tweezers when it is deployed.

11. The encased case of claim 10 wherein the positional plate has a front face and a back face, the front face having a triangularly curved elevation positioned at a desired angle along an outside surface of the positional plate, the elevation having a mole on top where an indentation on the tweezers lodges.

12. An encased tweezers, comprising:
a trough like hollow case having a hollow trough section constricting on one end, the hollow trough section partially concealing a body of the tweezers, leaving a top portion protruding beyond a top border of the hollow case when the tweezers is in a nested position, the protruding portion providing a grip space for deploying the tweezers from the hollow case;
a tweezers having a fused end and a pincer opposite the fused end, the fused end inserting between two opposite borders at the constricted end of the hollow case;
means for attaching the tweezers to the hollow case; and,
means for ejecting the tweezers from the hollow case.

13. The encased case of claim 12 wherein the means for ejecting the tweezers is a combination of a curved spring placed inside a bottom surface of the hollow case and a latch having a switching head on one end and a staging seat on the other end introduced into an opening with a recessed protruding portion at a bottom end of the hollow trough section, the recessed protruding portion engaging with the switching head of the latch as it traverse away from the constricted end of the hollow trough when a tip of the tweezers is rested on the staging seat as the tweezers press on the curved spring keeping the tweezers nested inside the hollow trough or towards the constricted end causing the staging seat to retract and release the tip of the tweezers and relax the curved spring to eject the tweezers from the hollow case.

14. The encased case of claim 12 further comprising a positional plate between an inside surface of one border and a lateral side of the tweezers or between adjacent lateral sides of two tweezers for positioning the tweezers when it is deployed.

15. The encased case of claim 15 wherein the positional plate has a front face and a back face, the front face having a triangularly curved elevation positioned at a desired angle along an outside surface of the positional plate, the elevation having a mole on top where an indentation on the tweezers lodges.

16. An encased tweezers, comprising:
a trough like hollow case having a hollow trough section constricting on one end, the hollow trough section partially concealing a body of the tweezers, leaving a top portion protruding beyond a top border of the hollow case when the tweezers is in a nested position, the protruding portion providing a grip space for deploying the tweezers from the hollow case;
a tweezers having a fused end and a pincer opposite the fused end, the fused end inserting between two opposite borders at the constricted end of the hollow case;
a curved spring placed inside a bottom surface of the hollow case;
a latch having a switching head on one end and a staging seat on the other end introduced into an opening with a recessed protruding portion at a bottom end of the hollow trough section, the recessed protruding portion engaging with the switching head of the latch as it traverse away from the constricted end of the hollow trough when a tip of the tweezers is rested on the staging seat as the tweezers press on the curved spring keeping the tweezers nested inside the hollow trough or towards the constricted end causing the staging seat to retract and release the tip of the tweezers and relax the curved spring to eject the tweezers from the hollow case; and,
means for attaching the tweezers to the hollow case.

17. The encased case of claim 16 further comprising a positional plate between an inside surface of one border and a lateral side of the tweezers or between adjacent lateral sides of two tweezers for positioning the tweezers when it is deployed.

18. The encased case of claim 17 wherein the positional plate has a front face and a back face, the front face having a triangularly curved elevation positioned at a desired angle along an outside surface of the positional plate, the elevation having a mole on top where an indentation on the tweezers lodges.

19. An encased tweezers, comprising:
a trough like hollow case having a hollow trough section constricting on one end, the hollow trough section partially concealing a body of the tweezers, leaving a top portion protruding beyond a top border of the hollow case when the tweezers is in a nested position, the protruding portion providing a grip space for deploying the tweezers from the hollow case;
a tweezers having a fused end and a pincer opposite the fused end, the fused end inserting between two opposite borders at the constricted end of the hollow case;
a positional plate between an inside surface of one border and a lateral side of the tweezers or between adjacent lateral sides of two tweezers for positioning the tweezers when it is deployed;
means for attaching the tweezers to the hollow case; and,
means for ejecting the tweezers from the hollow case.

20. The encased case of claim 19 wherein the positional plate has a front face and a back face, the front face having a triangularly curved elevation positioned at a desired angle along an outside surface of the positional plate, the elevation having a mole on top where an indentation on the tweezers lodges when the tweezers is deployed.
